(19) United States
(12) Patent Application Publication
(10) Pub. No.: US 2022/0122718 A1
(43) Pub. Date: Apr. 21, 2022

(54) Clinical Source of Truth with Patient Status Orders Automation and Validation throughout the Clinically Driven Revenue Cycle Management Life Cycle

(71) Applicant: CERNER INNOVATION, INC., Kansas City, KS (US)

(72) Inventors: Tanner Marvin, Lees Summit, MO (US); Jereme Wilson, Olathe, KS (US); Patricia Kilzer, Spartanburg, SC (US); Joshua Brasel, Kansas City, MO (US); Jody Taylor, Oviedo, FL (US)

(21) Appl. No.: 17/072,811

(22) Filed: Oct. 16, 2020

(51) Int. Cl. G16H 40/20; G06Q 40/00; G06Q 10/10; G06F 16/27; G06F 16/23

(52) U.S. Cl. CPC G16H 40/20; G06Q 40/12; G06F 16/2365; G06F 16/27; G06Q 10/10

(57) ABSTRACT

Methods, computer systems, and computer storage media are provided for facilitating claims management using planned orders. More particularly, encounter information can be synchronized with clinical system data in a revenue cycle management system so costly editing and delays can be avoided. Initially, an order for the patient during an encounter is received from a clinical system. A clinician may be prompted indicating the order cannot be completed because of a conflict. The revenue cycle management system monitors the clinical system for the missing information corresponding to the conflict. Once the missing information is received, the encounter information is synchronized with the missing information. The synchronized encounter information comprises automated edits that enable a claim corresponding to the encounter to be billable.

### BACKGROUND

The revenue cycle is a financial process utilized by health care systems to track revenue derived from patients. The process typically tracks a patient encounter from registration through the final payment of a balance. As such, the revenue cycle management system comprises many components, including preregistration, registration, charge capture, coding, claims submission, remittance processing, third-party follow, patient collections, utilization review, and the like. Current systems allow claims to be submitted for orders that may have missing information or mismatches in dates and/or times between certain aspects of a patient encounter. When the revenue cycle management system submits a claim, the claim is rejected and the opportunity for reimbursement is delayed or lost. In typical hospital systems, these scenarios account for 15-20% of all claim rejections within the revenue cycle management system. To address these rejections, current revenue cycle management systems require human intervention to manually review and edit data entries across multiple clinical systems to synchronize the data. These efforts are often time and cost prohibitive. As a result, the workforce necessary to intervene is overwhelmed and health care systems struggle to collect all potential revenue.

### BRIEF SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Embodiments of the present invention relate to leveraging clinical system data to optimize revenue cycle management. More particularly, the present invention utilizes clinical data to automatically synchronize encounter information with missing information such that a claim corresponding to an encounter is billable. A planned order for a patient is received at a revenue cycle management system. The revenue cycle management system monitors the clinical system for missing information corresponding to the conflict. Once the missing information is received, the encounter information is synchronized with the missing information. The synchronized encounter information comprises automated edits that enable a claim corresponding to the planned order to be billable.

### BRIEF DESCRIPTION OF THE SEVERAL VIEWS OF THE DRAWINGS

The present invention is described in detail below with reference to the attached drawing figures, wherein:

FIG. 1 is a block diagram of an exemplary computing environment suitable for use in implementing the present disclosure;

FIG. 2 is a block diagram of an exemplary system for providing clinically driven revenue cycle management, in accordance with an embodiment of the present disclosure;

FIG. 3 is a block diagram of an exemplary implementation of a patient status order (PSO) engine, in accordance with some embodiments of the present disclosure;

FIGS. 4-5 depict illustrative screen displays, in accordance with embodiments of the present disclosure;

FIG. 6 is a flow diagram showing an exemplary method for admitting a patient to inpatient from an emergency department utilizing a clinically driven revenue cycle management system, in accordance with various embodiments of the present disclosure;

FIG. 7 is a flow diagram showing an exemplary method for providing direct admit for a patient to inpatient utilizing a clinically driven revenue cycle management system, in accordance with various embodiments of the present disclosure;

FIGS. 8-11 depict illustrative screen displays, in accordance with embodiments of the present disclosure;

FIG. 12 is a flow diagram showing an exemplary method for providing adjustment orders utilizing a clinically driven revenue cycle management system, in accordance with various embodiments of the present disclosure;

FIGS. 13-16 depict illustrative screen displays, in accordance with embodiments of the present disclosure;

FIG. 17 is a flow diagram showing an exemplary method for providing clinical discharge utilizing a clinically driven revenue cycle management system, in accordance with various embodiments of the present disclosure;

FIGS. 18-19 depict illustrative screen displays, in accordance with embodiments of the present disclosure;

FIG. 20 is a flow diagram showing an exemplary method for providing clinically driven revenue cycle management, in accordance with various embodiments of the present disclosure.

### DETAILED DESCRIPTION

The subject matter of the present invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different components of methods employed, the terms should not be interpreted as implying any particular order among or between various steps herein disclosed unless and except when the order of individual steps is explicitly described.

As noted in the Background, current systems allow claims to be submitted for orders that may have missing information or mismatches in dates and/or times between certain aspects of a patient encounter. When the revenue cycle management system submits a claim, the claim is rejected and the opportunity for reimbursement is delayed or lost. In typical hospital systems, these scenarios account for 15-20% of all claim rejections within the revenue cycle management system. To address these rejections, current revenue cycle management systems require human intervention to manually review and edit data entries across multiple clinical systems to synchronize the data. These efforts are often time and cost prohibitive. As a result, the workforce necessary to intervene is overwhelmed and health care systems struggle to collect all potential revenue.

Embodiments of the present invention relate to leveraging clinical system data to optimize revenue cycle management. More particularly, the present invention utilizes clinical data to automatically synchronize encounter information with missing corresponding to a conflict such that a claim corresponding to an encounter is billable. The revenue cycle management system monitors the clinical system for missing information corresponding to the conflict. Once the missing information is received, the encounter information is synchronized with the missing information. The synchronized encounter information comprises automated edits that enable a claim corresponding to the encounter to be billable.

In embodiments, an order for the patient is initially received at the revenue cycle management system from the clinical system. A clinician may be prompted indicating the order cannot be completed because of a conflict. The revenue cycle converts the order into the planned order. The conflict may be missing information or a mismatch in data corresponding to the order. For example, the missing information may be registration information and the mismatch in data may be a mismatch in a day, time, or setting corresponding to the order.

Accordingly, in one aspect, an embodiment is directed to one or more computer storage media having computer-executable instructions embodied thereon that, when executed by a computer, causes the computer to perform operations. The operations include receiving, at a revenue cycle management system, a planned order for a patient that is not billable because of a conflict. The operations also includes monitoring, at the revenue cycle management system, one or more clinical systems for missing information corresponding to the conflict. The operations further include receiving, from the clinical system of the one or more clinical systems, the missing information corresponding to the conflict. The operations also include synchronizing the planned order with the missing information. The synchronized encounter information comprises automated edits that enable a claim corresponding to the planned order to be billable.

In another aspect of the invention, an embodiment of the present invention is directed to a computerized method. The method includes receiving, from a clinical system of the one or more clinical systems, an order for the patient. The method also includes causing a clinician to be prompted, at the clinical system of the one or more clinical systems. The prompt indicates that the order cannot be completed because of a conflict. The method further includes converting, at a revenue cycle management system, the order into a planned order for a patient. The method also includes monitoring, at the revenue cycle management system, the one or more clinical systems for additional information corresponding to the patient. The method further includes receiving, from the clinical system of the one or more clinical systems, the additional information corresponding to the patient. The method also includes synchronizing the planned order with the additional information. The synchronized encounter information comprises automated edits that enable a claim corresponding to the planned order to be billable.

In a further aspect, an embodiment is directed to a computerized system that includes one or more processors and a non-transitory computer storage medium storing computer-useable instructions that, when used by the one or more processors, cause the one or more processors to: receive, at a revenue cycle management system, a planned order for a patient that is not billable because of a conflict; monitor, at the revenue cycle management system, one or more clinical systems for missing information corresponding to the conflict; receive, from the clinical system of the one or more clinical systems, the missing information corresponding to the conflict; synchronize the planned order with the missing information, the synchronized encounter information comprising automated edits that enable a claim corresponding to the planned order to be billable.

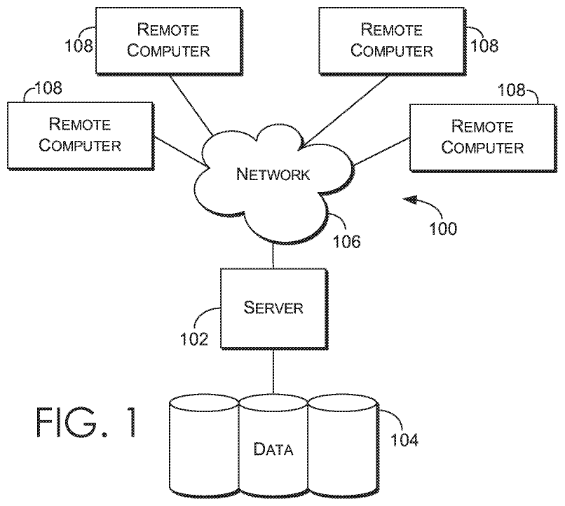

An exemplary computing environment suitable for use in implementing embodiments of the present invention is described below. FIG. 1 is an exemplary computing environment (e.g., medical-information computing-system environment) with which embodiments of the present invention may be implemented. The computing environment is illustrated and designated generally as reference numeral 100. The computing environment 100 is merely an example of one suitable computing environment and is not intended to suggest any limitation as to the scope of use or functionality of the invention. Neither should the computing environment 100 be interpreted as having any dependency or requirement relating to any single component or combination of components illustrated therein.

The present invention might be operational with numerous other purpose computing system environments or configurations. Examples of well-known computing systems, environments, and/or configurations that might be suitable for use with the present invention include personal computers, server computers, hand-held or laptop devices, multiprocessor systems, microprocessor-based systems, set top boxes, programmable consumer electronics, network PCs, minicomputers, mainframe computers, distributed computing environments that include any of the above-mentioned systems or devices, and the like.

The present invention might be described in the general context of computer-executable instructions, such as program modules, being executed by a computer. Exemplary program modules comprise routines, programs, objects, components, and data structures that perform particular tasks or implement particular abstract data types. The present invention might be practiced in distributed computing environments where tasks are performed by remote processing devices that are linked through a communications network. In a distributed computing environment, program modules might be located in association with local and/or remote computer storage media (e.g., memory storage devices).

With continued reference to FIG. 1, the computing environment 100 comprises a computing device in the form of a control server 102. Exemplary components of the control server 102 comprise a processing unit, internal system memory, and a suitable system bus for coupling various system components, including data store 104, with the control server 102. The system bus might be any of several types of bus structures, including a memory bus or memory controller, a peripheral bus, and a local bus, using any of a variety of bus architectures. Exemplary architectures comprise Industry Standard Architecture (ISA) bus, Micro Channel Architecture (MCA) bus, Enhanced ISA (EISA) bus, Video Electronic Standards Association (VESA) local bus, and Peripheral Component Interconnect (PCI) bus, also known as Mezzanine bus.

The control server 102 typically includes therein, or has access to, a variety of computer-readable media. Computer-readable media can be any available media that might be accessed by control server 102, and includes volatile and nonvolatile media, as well as, removable and nonremovable media. By way of example, and not limitation, computer-readable media may comprise computer storage media and communication media. Computer storage media includes both volatile and nonvolatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by control server 102. Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above should also be included within the scope of computer-readable media.

The control server 102 might operate in a computer network 106 using logical connections to one or more remote computers 108. Remote computers 108 might be located at a variety of locations in a medical or research environment, including clinical laboratories (e.g., molecular diagnostic laboratories), hospitals and other inpatient settings, ambulatory settings, medical billing and financial offices, hospital administration settings, home healthcare environments, clinicians' offices, Center for Disease Control, Centers for Medicare & Medicaid Services, World Health Organization, any governing body either foreign or domestic, Health Information Exchange, and any healthcare/government regulatory bodies not otherwise mentioned. Clinicians may comprise a treating physician or physicians; specialists such as intensivists, surgeons, radiologists, cardiologists, and oncologists; emergency medical technicians; physicians' assistants; nurse practitioners; nurses; nurses' aides; pharmacists; dieticians; microbiologists; laboratory experts; laboratory technologists; genetic counselors; researchers; students; and the like. The remote computers 108 might also be physically located in nontraditional medical care environments so that the entire healthcare community might be capable of integration on the network. The remote computers 108 might be personal computers, servers, routers, network PCs, peer devices, other common network nodes, or the like and might comprise some or all of the elements described above in relation to the control server 102. The devices can be personal digital assistants or other like devices.

Computer networks 106 comprise local area networks (LANs) and/or wide area networks (WANs). Such networking environments are commonplace in offices, enterprise-wide computer networks, intranets, and the Internet. When utilized in a WAN networking environment, the control server 102 might comprise a modem or other means for establishing communications over the WAN, such as the Internet. In a networking environment, program modules or portions thereof might be stored in association with the control server 102, the data store 104, or any of the remote computers 108. For example, various application programs may reside on the memory associated with any one or more of the remote computers 108. It will be appreciated by those of ordinary skill in the art that the network connections shown are exemplary and other means of establishing a communications link between the computers (e.g., control server 102 and remote computers 108) might be utilized.

In operation, an organization might enter commands and information into the control server 102 or convey the commands and information to the control server 102 via one or more of the remote computers 108 through input devices, such as a keyboard, a pointing device (commonly referred to as a mouse), a trackball, or a touch pad. Other input devices comprise microphones, satellite dishes, scanners, or the like. Commands and information might also be sent directly from a remote healthcare device to the control server 102. In addition to a monitor, the control server 102 and/or remote computers 108 might comprise other peripheral output devices, such as speakers and a printer.

Although many other internal components of the control server 102 and the remote computers 108 are not shown, such components and their interconnection are well known. Accordingly, additional details concerning the internal construction of the control server 102 and the remote computers 108 are not further disclosed herein.

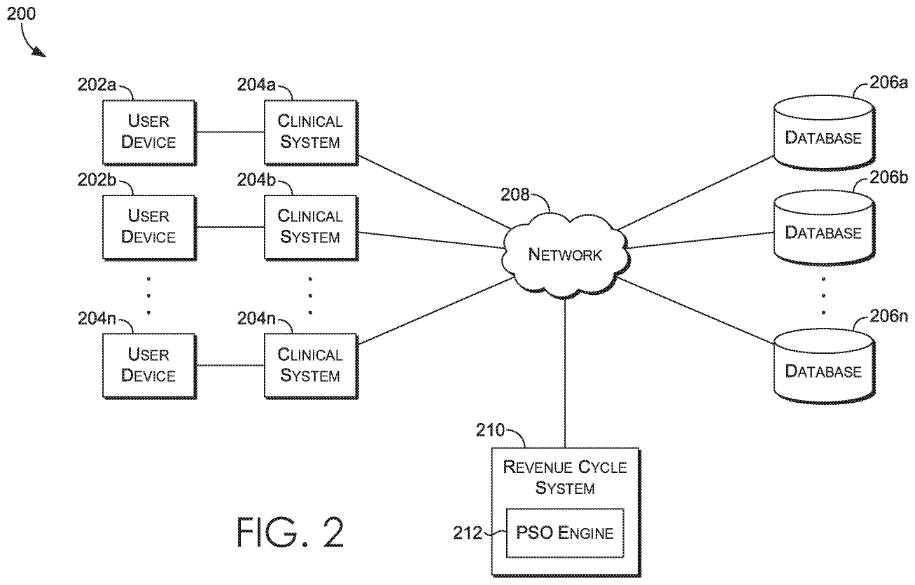

Turning now to FIG. 2, an exemplary clinically driven revenue cycle management system 200 is depicted suitable for use in implementing embodiments of the present invention. The clinically driven revenue cycle management system 200 is merely an example of one suitable computing system environment and is not intended to suggest any limitation as to the scope of use or functionality of embodiments of the present invention. Neither should the revenue cycle management system 200 be interpreted as having any dependency or requirement related to any single module/component or combination of modules/components illustrated therein.

The clinically driven revenue cycle management system 200 includes user device(s) 202a-202n, clinical system(s) 204a-204n, database(s) 206a-206n, and revenue cycle management system 210, all in communication with one another via a network 208. The network 208 may include, without limitation, one or more secure local area networks (LANs) or wide area networks (WANs). The network 208 may be a secure network associated with a facility such as a healthcare facility. The secure network may require that a user log in and be authenticated in order to send and/or receive information over the network.

The components/modules illustrated in FIG. 2 are exemplary in nature and in number and should not be construed as limiting. Any number of components/modules may be employed to achieve the desired functionality within the scope of embodiments hereof. Further, components/modules may be located on any number of servers. By way of example only, revenue cycle management system 210 might reside on a server, cluster of servers, or a computing device remote from one or more of the remaining components. Although illustrated as separate systems, the functionality provided by each of these components might be provided as a single component/module. The single unit depictions are meant for clarity, not to limit the scope of embodiments in any form.

Components of the clinically driven revenue cycle management system 200 may include a processing unit, internal system memory, and a suitable system bus for coupling various system components, including one or more data stores for storing information (e.g., files and metadata associated therewith). Components of the clinically driven revenue cycle management system 200 typically includes, or has access to, a variety of computer-readable media.

It should be understood that this and other arrangements described herein are set forth only as examples. Other arrangements and elements (e.g., machines, interfaces, functions, orders, and groupings of functions, etc.) can be used in addition to or instead of those shown, and some elements may be omitted altogether. Further, many of the elements described herein are functional entities that may be implemented as discrete or distributed components or in conjunction with other components/modules, and in any suitable combination and location. Various functions described herein as being performed by one or more entities may be carried out by hardware, firmware, and/or software. For instance, various functions may be carried out by a processor executing instructions stored in memory.

Each of clinical system 204a-204n includes or has access to infrastructure that is capable of receiving and communicating information for use by, for example, revenue cycle management system 210. The information received and communicated in association with each of clinical system 204a-204n may comprise general information used by revenue cycle management system 210. Each of clinical system 204a-204n may receive data from user device(s) 202a-202n or other systems (e.g., disparate healthcare systems), which may include any number or type of medical devices that may be utilized to provide or measure patient care to a patient.

Each of clinical system 204a-204n includes or has access to infrastructure that is capable of storing electronic health records (EHRs), such as database(s) 206a-206n of patients associated with clinical system(s) 204a-204n. EHRs may comprise electronic clinical documents such as images, clinical notes, orders, summaries, reports, analyses, or other types of electronic medical documentation relevant to a particular patient's condition and/or treatment. Electronic clinical documents contain various types of information relevant to the condition and/or treatment of a particular patient and can include information relating to, for example, patient identification information, images, alert history, culture results, physical examinations, vital signs, past medical histories, surgical histories, family histories, histories of present illnesses, current and past medications, allergies, symptoms, past orders, completed orders, pending orders, tasks, lab results, other test results, patient encounters and/or visits, immunizations, physician comments, nurse comments, other caretaker comments, and a host of other relevant clinical information. In some embodiments, clinical system 204a-204n may receive data from health information exchanges (“HIEs”), personal health records (“PHRs”), patient claims, and other health records associated with a patient.

User device(s) 202a-202n may be any type of computing device used within a healthcare facility or as part of the claims processing process to receive, display, and send information to another user or system. User device(s) 202a-202n may be capable of communicating via the network with clinical system(s) 204a-204n, database(s) 206a-206n, or revenue cycle management system 210. Such devices may include any type of mobile and portable devices including cellular telephones, personal digital assistants, tablet PCs, smart phones, and the like.

User device(s) 202a-202n is configured to display information to a clinician or user via a display. The information may include communications initiated by and/or received by revenue cycle management system 210. Embodiments are not intended to be limited to visual display but rather may also include audio presentation, visual presentation, combined audio/visual presentation, and the like.

Generally, the revenue cycle management system 210 is configured to track patient encounters for a client (e.g., clinical system(s) 204a-204n) from preregistration through the final payment of a balance. For example, the revenue cycle management system 210 tracks and collects data for patient encounters for each clinical system 204a-204n at preregistration, registration, charge capture, coding, claims submission, remittance processing, third-party follow, patient collections, utilization review, and the like. Although the revenue cycle management system 210 is depicted as a single system, it is contemplated that each clinical system 204a-204n may utilize a different revenue cycle management system 210. As shown in FIG. 2, revenue cycle management system 210 includes a PSO engine 212, described in more detail below.

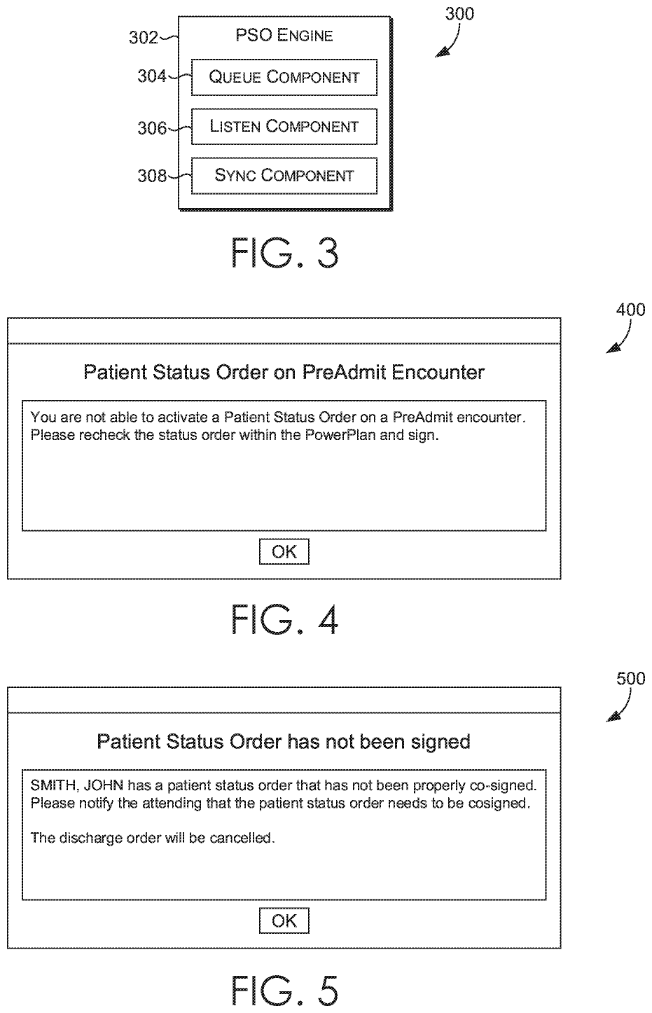

Referring now to FIG. 3, the PSO engine 300 includes several components. For example, the PSO engine 300 may include a queue component 302, a listen component 304, and a sync component 306. Initially, the queue component 302 receives, from a clinical system, an order for a patient during an encounter. Queue component 302 may cause a clinician to be prompted, such as via a user device, at the clinical system. The prompt indicates that the order cannot be completed because of a conflict with encounter information corresponding to the encounter. Additionally or alternatively, queue component 302 may cause an alert to be provided, such as via the user device, at the clinical system. The alert may indicate that the patient needs to be fully registered or a reason the order cannot be completed or billed.

Next, the listen component 304 monitors, at the revenue cycle management system, the clinical systems for additional information corresponding to the conflict. When the listen component 304 identifies the additional information, the additional information is received form the clinical system.

Finally, the sync component 306 synchronizes the encounter information with the missing information. The synchronized encounter information comprises automated edits that enable a claim corresponding to the encounter to be billable.

In FIGS. 4 and 5, displays 400 and 500 illustrate various conflicts in accordance with embodiments of the present disclosure. In FIG. 4, the display 400 provides an alert to the user that the user cannot activate an order for a patient that only has preadmit encounter information. In FIG. 5, the display provides an alert to the user that a discharge order for a patient has not been properly co-signed and the discharge order will be cancelled. Accordingly, the clinician may enter a hold order or a planned order. Once the encounter information has been fully provided or the discharge order has been properly co-signed, the encounter will be synchronized with the hold order or the planned order, and a claim corresponding to the encounter can be billed.

Other conflicts may include: inpatient admit date and time cannot be before the pre-registration date and time, inpatient admit date and time cannot be before the registration date and time, inpatient admit date and time cannot be after the clinical discharge date and time, inpatient admit date and time cannot be after the discharge date and time, the admit decision date time cannot be after the inpatient admit date time, the outpatient in bed date and time cannot be after the inpatient admit date and time, the observation start date and time cannot be after the inpatient admit date and time, the arrival date time cannot be after the inpatient admit date time, outpatient in bed date and time cannot be after the observation start date and time, the observation start date and time cannot be after the inpatient admit date and time, the observation start date and time cannot be after the clinical discharge date and time, the observation start date and time cannot be after the discharge date and time, discharge date and time must not be before the arrival date and time, or the discharge date and time must not be before the registration date and time.

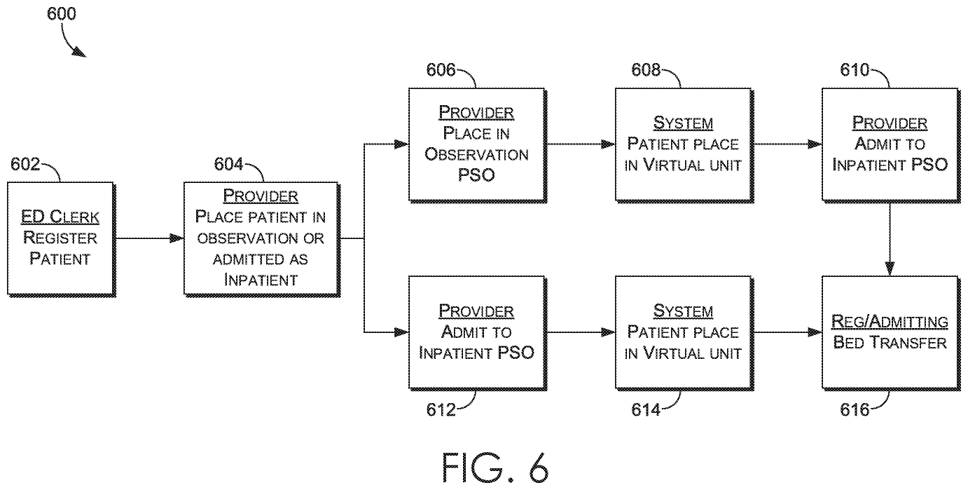

Referring to FIG. 6, a flow diagram is provided illustrating a method 600 for providing showing an exemplary method for admitting a patient to inpatient from an emergency department utilizing a clinically driven revenue cycle management system, in accordance with various embodiments of the present disclosure. Method 600 may be performed by any computing device (such as computing device described with respect to FIG. 1) with access to a revenue cycle management system (such as the one described with respect to FIG. 2) or by one or more components of the revenue cycle management system (such as the PSO engine described with respect to FIGS. 2 and 3).

Initially, as shown at step 602, an emergency department clerk registers a patient that has not yet presented at the emergency department. For example, the emergency department clerk may register the patient using information already known about the patient (such as from previous visits within the healthcare system). At step 604, a provider determines whether to place the patient in observation or admits the patient as an inpatient.

At this point, if the providers tries to place the patient in observation via a conventional order, an error is generated indicating the patient has not been registered yet. Instead, the provider places the patient in observation via an observation PSO (i.e., a planned order), as shown at step 606. Accordingly, the revenue cycle management system places the patient in a virtual unit, as shown at step 608. Next, the provider may determine to admit the patient via an inpatient PSO, as shown at step 610. Once the patient is registered and admitted, the system synchronizes the registration data (i.e., the encounter information) with additional information from the PSO (such as bed transfer information, patient type, accommodation, medical service, and/or admitting and attending physicians), as shown at step 616, and a claim corresponding to the encounter can be billed.

If, on the other hand, the provider, at the outset, attempts to admit the patient via an order, an error is generated indicating the patient has not been registered yet. Instead, the provider admits the patient via an inpatient PSO, as shown at step 612, and the revenue cycle management system places the patient in a virtual unit, as shown at step 614. Again, once the patient is registered and admitted, the system synchronizes the registration data with additional information from the PSO (such as bed transfer information, patient type, accommodation, medical service, and/or admitting and attending physicians), as shown at step 616, and a claim corresponding to the encounter can be billed.

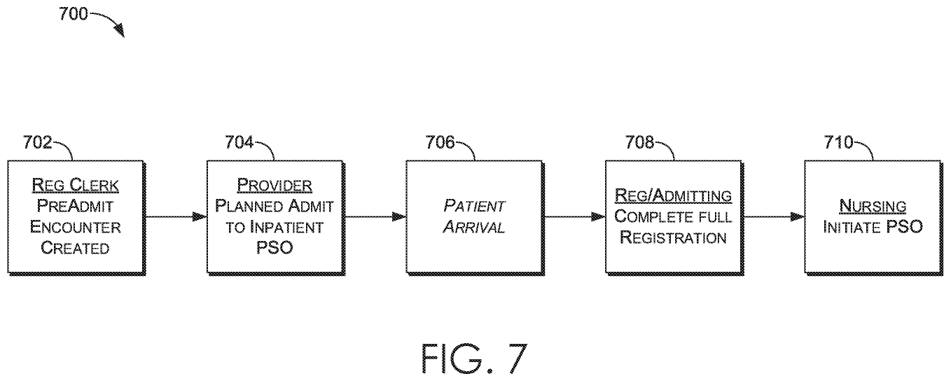

In FIG. 7, a flow diagram is provided illustrating a method 700 for providing direct admit for a patient to inpatient utilizing a clinically driven revenue cycle management system, in accordance with various embodiments of the present disclosure. Method 700 may be performed by any computing device (such as computing device described with respect to FIG. 1) with access to a revenue cycle management system (such as the one described with respect to FIG. 2) or by one or more components of the revenue cycle management system (such as the PSO engine described with respect to FIGS. 2 and 3).

Initially, as shown at step 702, a registration clerk creates a preadmit encounter. To do so, the registration clerk may enter information such as patient type, accommodation, medical service, and/or admitting and attending physicians. At step 704, a provider determines whether to move the planned admit to an inpatient. At this point, if the provider tries to place the patient in inpatient via a conventional order, an error is generated indicating the patient has not arrived or been fully registered yet. Instead, the provider places the patient in inpatient via an inpatient PSO (i.e., a planned order). Accordingly, when the patient arrives, as shown at step 706, the registration clerk can complete full registration, at step 708. When a nurse, or other clinician, initiates the PSO, as shown at step 708, the initiated PSO fields overwrite values entered at the time of full registration and claim corresponding to the encounter can be billed.

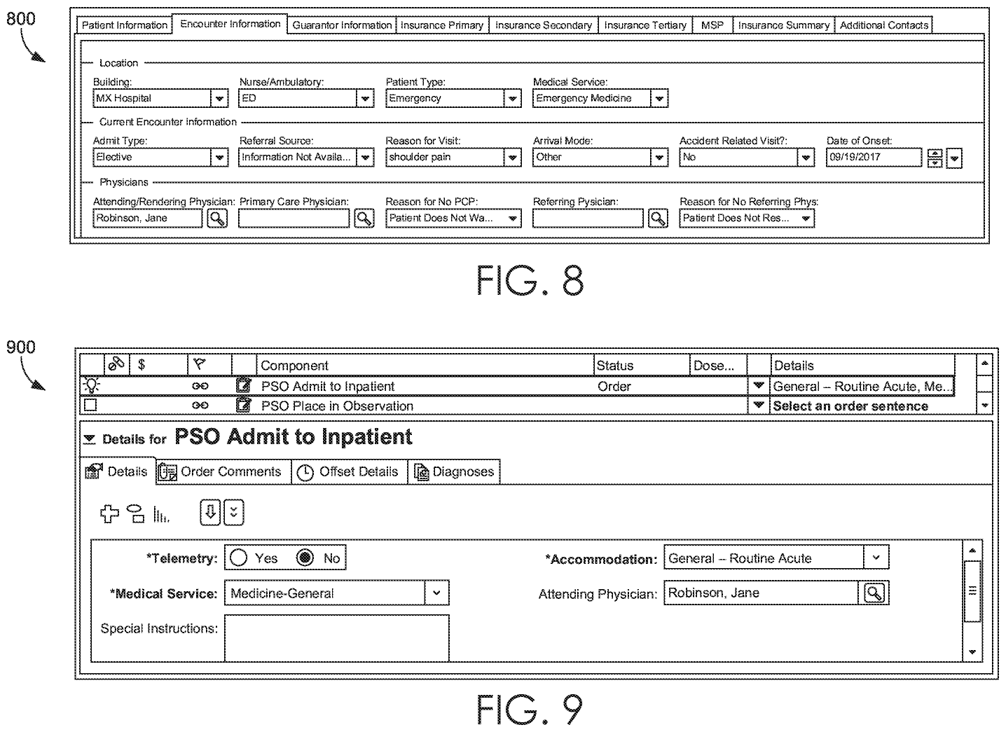

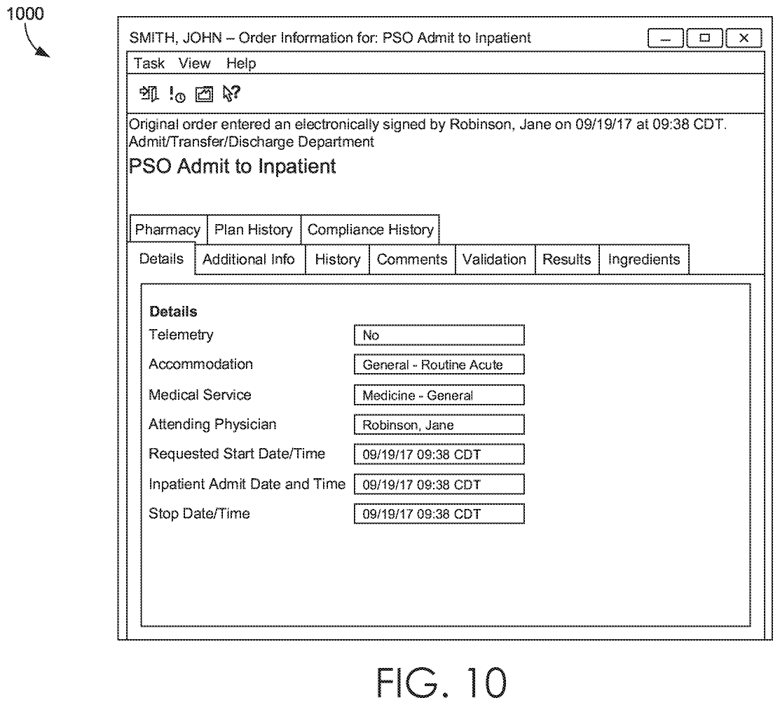

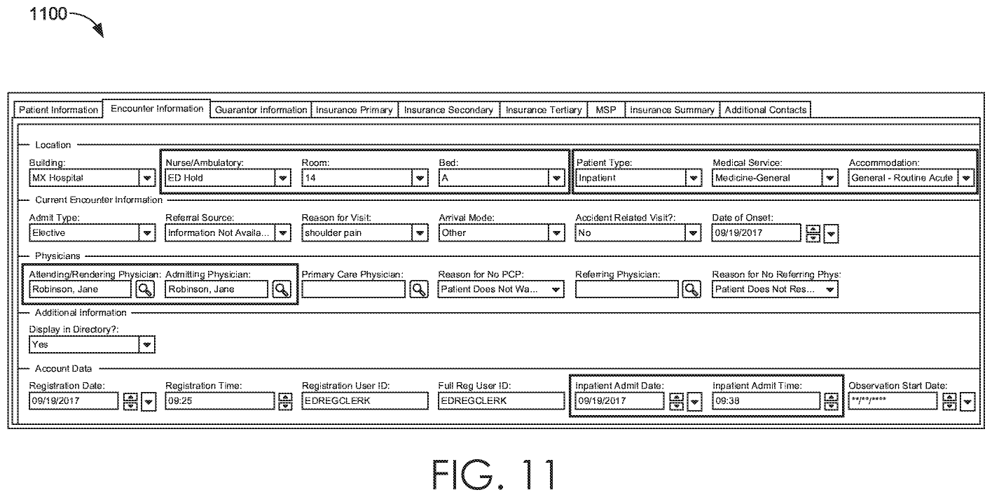

As shown in FIGS. 8-11, displays 800-1100 illustrate screen displays, in accordance with various embodiments of the present disclosure. More particularly, displays 800-1100 illustrate synchronizing encounter information with information provided in PSOs. Initially, display 800 illustrates initial encounter information being received prior to patient arrival (i.e., using information already known about the patient such as from previous visits within the healthcare system) or upon a patient first presenting at the emergency department.

Referring to display 900, a clinician may determine to initiate a PSO for the patient to admit the patient to inpatient or place in observation. In display 1000, the PSO includes information such as, but not limited to, encounter type, medical service and accommodation, location (for example, if the previous location was the emergency department, the location may be updated to virtual unit), ordering physician may be updated to admitting physician, attending physician, and/or inpatient admit date/time if the patient is admitted to inpatient or observation start date/time if the patient is placed in observation. As shown in display 1100, the initial encounter information is synchronized with information from the PSO, enabling a claim corresponding to the encounter to be billable.

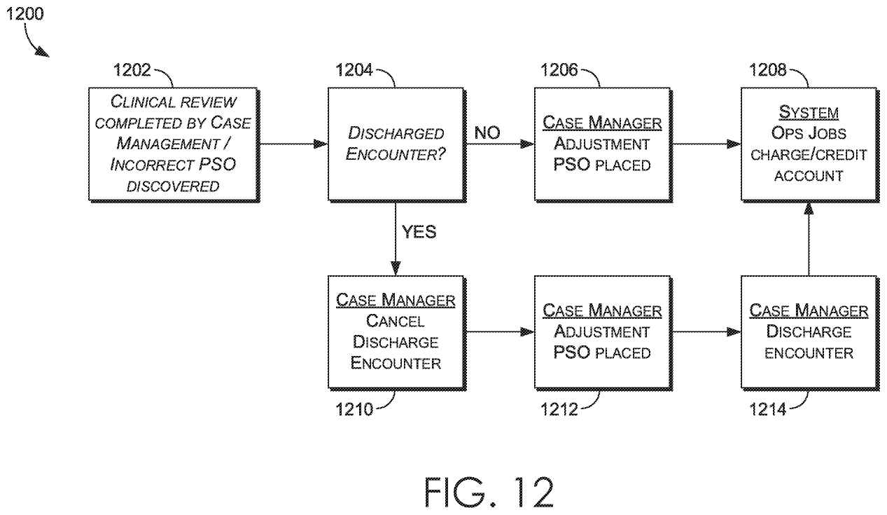

Turning now to FIG. 12, a flow diagram is provided illustrating a method 1200 for providing adjustment orders utilizing a clinically driven revenue cycle management system, in accordance with various embodiments of the present disclosure. Method 1200 may be performed by any computing device (such as computing device described with respect to FIG. 1) with access to a revenue cycle management system (such as the one described with respect to FIG. 2) or by one or more components of the revenue cycle management system (such as the PSO engine described with respect to FIGS. 2 and 3).

Initially, as shown at step 1202, clinical review may be completed by case management. During this process an incorrect PSO may be discovered. At step 1204, it is determined whether the incorrect PSO is a discharged encounter. If it is not a discharged encounter, at step 1206, an adjustment PSO can be placed by the case manager. The revenue cycle management system charges or credits the account in accordance with the adjustment PSO, at step 1208.

If on the other hand, at step 1204, it is determined the incorrect PSO is a discharged encounter, the case manager cancels the discharged encounter at step 1210. Next, an adjustment PSO can be placed, at step 1212, by the case manager. At step 1214, the case manager discharges the encounter and the revenue cycle management system charges or credits the account in accordance with the adjustment PSO, at step 1208.

In embodiments, adjustment orders overwrite previous order information. For example, adjustment orders can overwrite encounter types, accommodations, and the like. If the patient is a Medicare patient, an adjustment order cannot be placed.

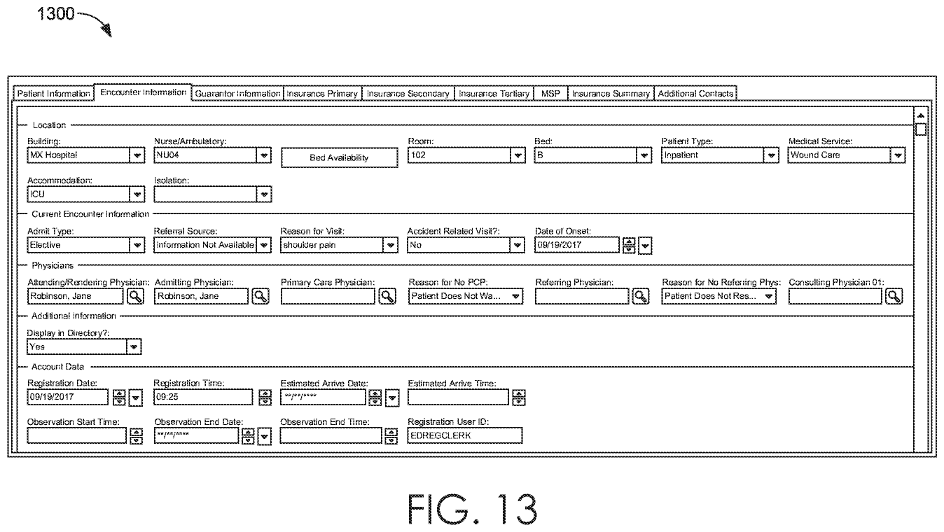

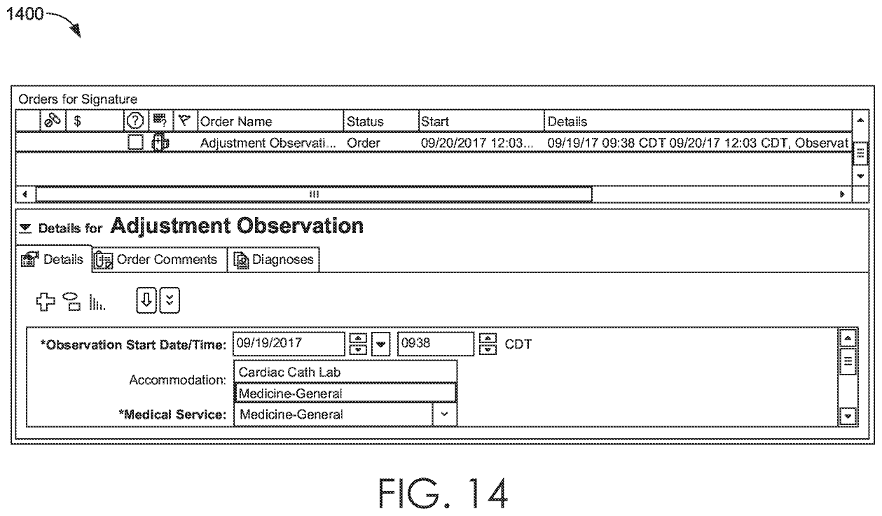

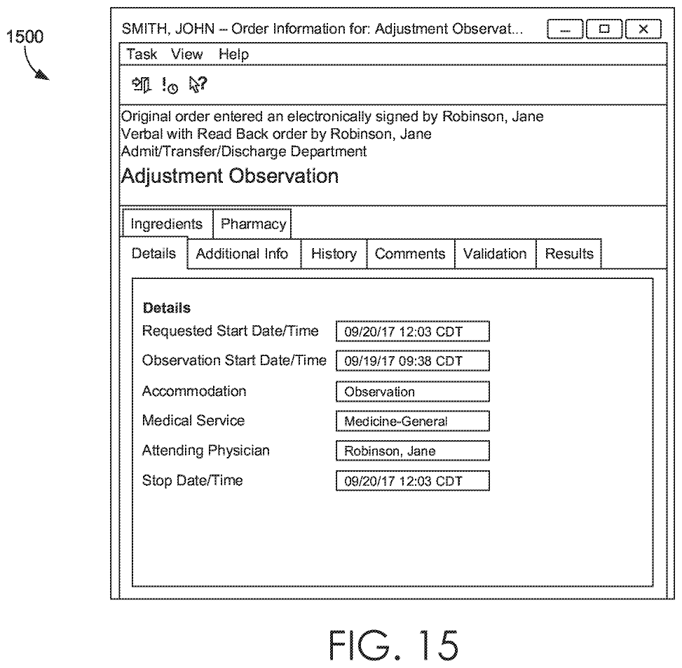

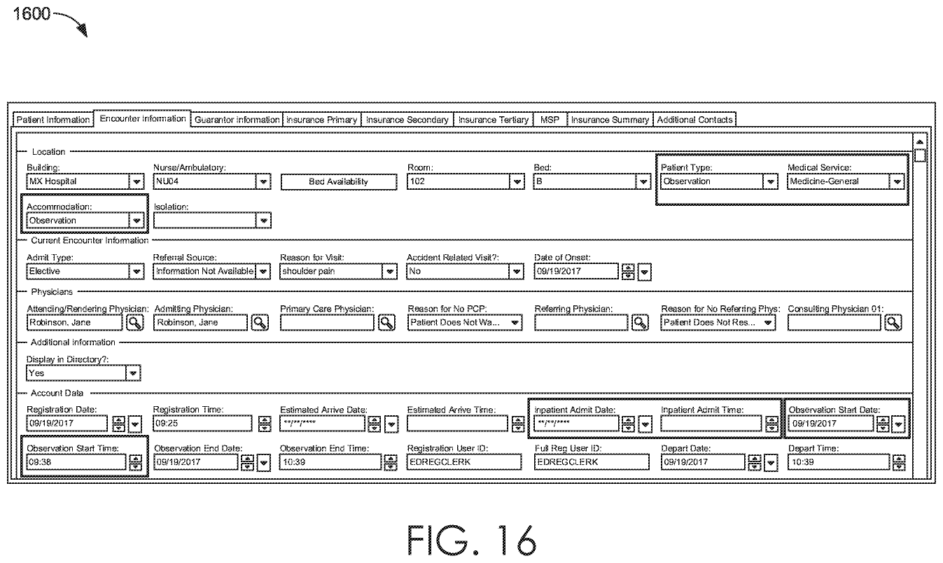

In FIGS. 13-16, displays 1300-1600 illustrate screen displays, in accordance with various embodiments of the present disclosure. More particularly, displays 1300-1600 illustrate providing adjustment orders utilizing a clinically driven revenue cycle management system. Initially, display 1300 illustrates initial encounter information being received prior to patient arrival (i.e., using information already known about the patient such as from previous visits within the healthcare system) or upon a patient first presenting at the emergency department.

Referring to display 1400, a clinician may determine to enter an adjustment order. For example, the clinician may adjust the patient type, the medical service and/or accommodation, an inpatient admit date/time, or an observation start date/time. As shown in display 1500, an adjustment order is entered to provide details for a patient in observation. In this example, the adjustment order changes the observation start date/time from the requested start date/time. Additionally, the observation end date/time, accommodation, and medical service can also be adjusted. Any adjustments are synchronized with the initial encounter information, as shown in display 1600, and a claim corresponding to the encounter can be billed.

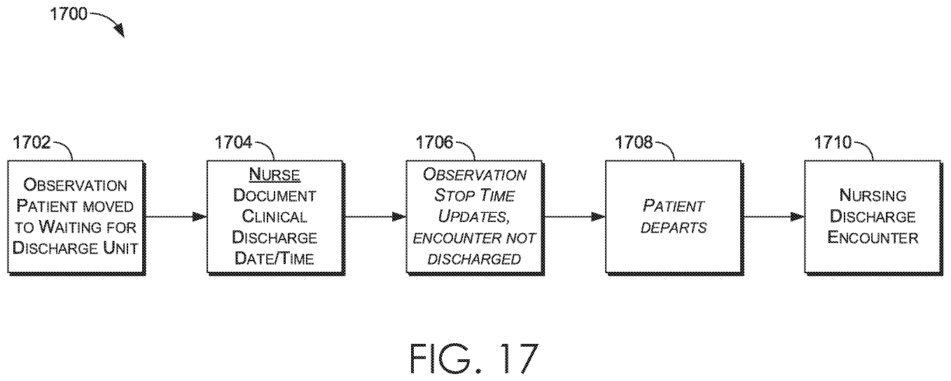

Referring next to FIG. 17, a flow diagram is provided illustrating a method 1700 for providing encounter updates, in accordance with various embodiments of the present disclosure. More particularly, method 1700 provides clinical discharge utilizing a clinically driven revenue cycle management system. Method 1700 may be performed by any computing device (such as computing device described with respect to FIG. 1) with access to a revenue cycle management system (such as the one described with respect to FIG. 2) or by one or more components of the revenue cycle management system (such as the PSO engine described with respect to FIGS. 2 and 3).

Initially, at step 1702, an observation patient is moved to a waiting for discharge unit. At step 1704, a nurse documents the patient has been moved to clinical discharge at a particular date/time. Updates to the observation stop time are provided at step 1706. At this point the encounter is not discharged. At step 1708, the patient departs and the nursing discharges the encounter, at step 1710.

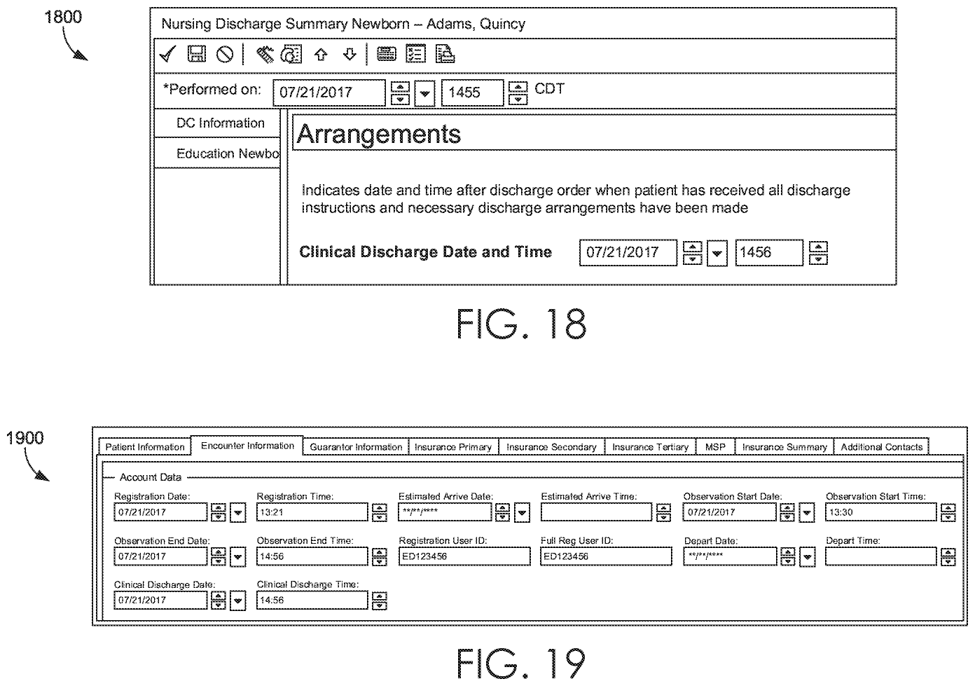

In FIGS. 18 and 19, displays 1800, 1900 illustrate screen displays, in accordance with various embodiments of the present disclosure. More particularly, displays 1800, 1900 illustrate a method for providing encounter updates, in accordance with various embodiments of the present disclosure. Initially, display 1800 illustrates a PSO for a clinical discharge. As shown, a nurse documents a date/time after discharge instructions have been provided and discharge arrangements have been made. Accordingly, the encounter is updated, as shown in display 1900, with the clinical discharge information from the PSO when the patient is physically discharged. This enables the nurse to discharge the encounter and a claim corresponding to the encounter can be billed.

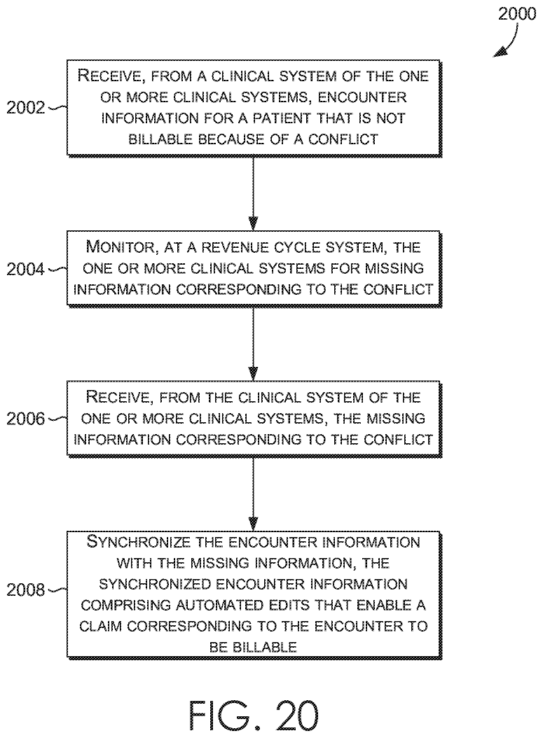

Referring now to FIG. 20 a flow diagram is provided illustrating a method 2200 for providing clinically driven revenue cycle management, in accordance with an embodiment of the present invention. Method 2000 may be performed by any computing device (such as computing device described with respect to FIG. 1) with access to a revenue cycle management system (such as the one described with respect to FIG. 2) or by one or more components of the revenue cycle management system (such as the PSO engine described with respect to FIGS. 2 and 3).

As shown at step 2002, encounter information is received, at a revenue cycle management system, for a patient that is not billable because of a conflict. In some embodiments, an order for the patient is initially received from a clinical system of the one or more clinical system. This may cause a clinician to be prompted, at the clinical system of the one or more clinical systems. The prompt indicates that the order cannot be completed because of the conflict. In some embodiments, an alert is communicated to the clinical system, indicating why the order cannot be completed. Based on the conflict, the order is converted, at the revenue cycle management system, into the planned order.

At step 2004, one or more clinical systems are monitored, at the revenue cycle management system, for missing information corresponding to the conflict. In some embodiments, the conflict represents the missing information or a mismatch in data corresponding to the order. For example, the missing information may be registration information. In another example, the mismatch in data corresponding to the order may comprise a mismatch in a day or a time corresponding to the order or a mismatch in a setting corresponding to the order. In embodiments, the setting comprises inpatient, outpatient, observation, discharge, or clinical discharge.

At step 2006, the missing information corresponding to the conflict is received, from the clinical system of the one or more clinical systems. At step 2008, the encounter information is synchronized with the missing information. The synchronized encounter information comprises automated edits that enable a claim corresponding to the planned order to be billable.

With reference to FIGS. 4-5, 8-11, 13-16, and 18-19 illustrative screen displays 400-500, 800-1100, 1300-1600, and 1800-1900 of embodiments of the present invention are shown. It is understood that each of the illustrative screen displays are connected logically, such that they comprise a user interface designed for providing clinically driven revenue cycle management. The screen displays may appear in any order and with any number of screen displays, without regard to whether the screen display is described or depicted herein. The screen displays provide tools that enable clinically driven revenue cycle management in accordance with embodiments of the present invention.

As can be understood, the present invention provides systems, methods, and user interfaces for providing clinically driven revenue cycle management. The present invention has been described in relation to particular embodiments, which are intended in all respects to be illustrative rather than restrictive. Alternative embodiments will become apparent to those of ordinary skill in the art to which the present invention pertains without departing from its scope.

From the foregoing, it will be seen that this invention is one well adapted to attain all the ends and objects set forth above, together with other advantages which are obvious and inherent to the system and method. It will be understood that certain features and subcombinations are of utility and may be employed without reference to other features and subcombinations. This is contemplated and within the scope of the claims.

What is claimed is:

1. One or more computer storage media having computer-executable instructions embodied thereon, that when executed, perform operations, the operations comprising:
receiving, at a revenue cycle management system, encounter information for a patient that is not billable because of a conflict;
monitoring, at the revenue cycle management system, one or more clinical systems for missing information corresponding to the conflict;
receiving, from the clinical system of the one or more clinical systems, the missing information corresponding to the conflict;
synchronizing the encounter information with the missing information, the synchronized encounter comprising automated edits that enable a claim corresponding to the encounter to be billable.

2. The media of claim 1, further comprising receiving, from a clinical system of the one or more clinical systems, an order for the patient.

3. The media of claim 2, further comprising causing a clinician to be prompted, at the clinical system of the one or more clinical systems, the prompt indicating that the order cannot be completed because of the conflict.

4. The media of claim 3, further comprising converting, at the revenue cycle management system, the order into the planned order that prompts the clinician for the missing information.

5. The media of claim 2, wherein the conflict represents the missing information or a mismatch in data corresponding to the order.

6. The media of claim 1, wherein the missing information is registration information.

7. The media of claim 5, wherein the mismatch in data corresponding to the order comprises a mismatch in a day or a time corresponding to the order or a mismatch in a setting corresponding to the order.

8. The media of claim 7, wherein the setting comprises inpatient, outpatient, observation, discharge, or clinical discharge.

9. The media of claim 2, further comprising communicating an alert, to the clinical system, that indicates why the order cannot be completed.

10. A computerized method comprising:
receiving, from a clinical system of the one or more clinical systems, an order for a patient during an encounter;
causing a clinician to be prompted, at the clinical system of the one or more clinical systems, the prompt indicating that the order cannot be completed because of a conflict with encounter information corresponding to the encounter;
monitoring, at the revenue cycle management system, the one or more clinical systems for additional information corresponding to the conflict;
receiving, from the clinical system of the one or more clinical systems, the additional information corresponding to the conflict;
synchronizing the encounter information with the additional information, the synchronized encounter information comprising automated edits that enable a claim corresponding to the encounter to be billable.

11. The method of claim 10, wherein the conflict represents missing information or a mismatch in data corresponding to the order.

12. The method of claim 11, wherein the missing information is registration information.

13. The method of claim 11, wherein the mismatch in data corresponding to the order comprises a mismatch in a day or a time corresponding to the order or a mismatch in a setting corresponding to the order.

14. The method of claim 13, wherein the setting comprises inpatient, outpatient, observation, discharge, or clinical discharge.

15. The method of claim 10, further comprising causing an alert, at the clinical system, to register the patient.

16. A computerized system comprising:
one or more processors; and
a non-transitory computer storage media storing computer-useable instructions that, when used by the one or more processors, cause the one or more processors to:
receive, at a revenue cycle management system, encounter information for a patient that is not billable because of a conflict;
monitor, at the revenue cycle management system, one or more clinical systems for missing information corresponding to the conflict;
receive, from the clinical system of the one or more clinical systems, the missing information corresponding to the conflict;
synchronize the encounter information with the missing information, the synchronized encounter information comprising automated edits that enable a claim corresponding to the encounter to be billable.

17. The computerized system of claim 16, further comprising receiving, from a clinical system of the one or more clinical systems, an order for the patient.

18. The computerized system of claim 17, further comprising causing a clinician to be prompted, at the clinical system of the one or more clinical systems, the prompt indicating that the order cannot be completed because of the conflict.

19. The computerized system of claim 18, further comprising converting, at the revenue cycle management system, the order into a planned order that prompts a user for the missing information.

20. The computerized system of claim 19, further comprising communicating an alert, to the clinical system, that indicates a reason the order cannot be completed.
